(19) United States
(12) Patent Application Publication
(10) Pub. No.: US 2021/0030713 A1
(43) Pub. Date: Feb. 4, 2021

(54) ANTHOCYANIDIN COMPLEX

(71) Applicant: Sapiotec GmbH, Wurzburg (DE)

(72) Inventors: Norbert Roewer, Wurzburg (DE); Jens Broscheit, Wurzburg (DE)

(21) Appl. No.: 16/876,539

(22) Filed: May 18, 2020

Related U.S. Application Data
(63) Continuation of application No. 15/120,670, filed on Aug. 22, 2016, now abandoned.

(30) Foreign Application Priority Data
Feb. 28, 2014 (EP) 14157248.7

(51) Int. Cl. A61K 31/353; C08B 37/16; C09B 61/00; A61K 47/69

(52) U.S. Cl. CPC A61K 31/353; A61K 47/6951; C09B 61/00; C08B 37/0015

(57) ABSTRACT

The invention relates to a complex of an anthocyanidin and a methylated β-cyclodextrin which can be formulated as an aqueous solution and as a solid, and to a process for the preparation of such a complex. Complexes according to the invention are storage-stable and can be readily formulated in aqueous solution.

The present application is a continuation of U.S. patent application Ser. No. 15/120,670, filed Aug. 22, 2016, which is a § 371 US National Entry of International Application No. PCT/EP2015/054084, filed Feb. 26, 2015.

The invention relates to a complex of an anthocyanidin and a cyclodextrin.

Anthocyanidins are zymochromic pigments which occur in most higher terrestrial plants. Anthocyanidins are sugar-free (aglycones) and closely related to the sugar-containing anthocyanins. Anthocyanidins are pigments and possess antioxidant properties.

Complexing of anthocyanidins with sulfoalkylether-β-cyclodextrins is already known from WO 2013/144297 A1.

The object underlying the invention is to provide anthocyanidins in a relatively concentrated form in which they are easy to handle and formulate and are storage-stable.

This object is achieved by a complex of an anthocyanidin and a methylated β-cyclodextrin.

Some terms used within the context of the invention will first be explained.

Anthocyanidins have the basic structure shown below.

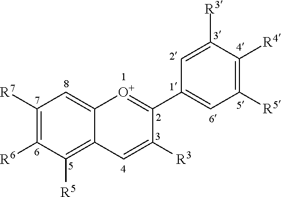

The substituents in this formula are selected from the group consisting of hydrogen, hydroxy group and methoxy group.

Cyclodextrins are cyclic oligosaccharides of glucose molecules linked by an α-1,4-glycosidic bond. β-Cyclodextrin possesses seven glucose units. In the case of a methylated β-cyclodextrin, hydroxy groups of the glucose unit are provided with methyl groups. According to the invention, generally only some of the 21 hydroxy groups of a β-cyclodextrin are methylated.

The preparation of methylated β-cyclodextrins is known to the person skilled in the art, appropriate products being obtainable, for example from Wacker Chemie, under the name Cavasol®.

The invention has recognized, surprisingly, that anthocyanidins such as delphinidin can complex at a much higher concentration with methylated β-cyclodextrins than with other cyclodextrins from the prior art. This is particularly surprising, therefore, since complexing experiments with a series of cyclodextrins were carried out, for example in WO 2013/144297 A1, in which the concentration or loading of the complex with anthocyanidin was orders of magnitude lower.

The present invention therefore makes it possible to provide anthocyanidins in high concentration and thus high dosages, preferably in aqueous or water-soluble form, and therefore to make accessible in a simple manner an in vivo administration, for example an i.v. administration. Furthermore, the invention is of particular advantage in that methylated cyclodextrins, in particular RAMEB, at the concentrations used, are non-toxic or at most minimally toxic and particularly in vivo trigger at most a minimal and harmless hemolysis, or none at all.

The degree of substitution of the β-cyclodextrin with methyl groups is preferably from 10 to 15, preferably from 11 to 14, more preferably from 12 to 13.

Particular preference is given to using RAMEB (randomly methylated β-cyclodextrin) as methylated β-cyclodextrin. It is a randomly methylated β-cyclodextrin having a degree of substitution of about 1.8 methyl groups per sugar unit or 12.5 methyl groups per cyclodextrin ring (DS degree of substitution approximately 12.5). RAMEB is commercially available, for example from Wacker Chemie, under the name Cavasol® W7 M Pharma.

The anthocyanidins complexed according to the invention are preferably selected from the group consisting of aurantinidin, cyanidin, delphinidin, europinidin, luteolinidin, pelargonidin, malvidin, peonidin, petunidin and rosinidin. The chemical structure corresponds to formula I given above with the following substitution pattern

| | R^{3′} | R^{4′} | R^{5′} | R³ | R⁵ | R⁶ | R⁷ |
|---|---|---|---|---|---|---|---|
| Aurantinidin | —H | —OH | —H | —OH | —OH | —OH | —OH |
| Cyanidin | —OH | —OH | —H | —OH | —OH | —H | —OH |

| | | | | | | | |
|---|---|---|---|---|---|---|---|
| Delphinidin | —OH | —OH | —OH | —OH | —OH | —H | —OH |
| Europinidin | —OCH₃ | —OH | —OH | —OH | —OCH₃ | —H | —OH |
| Luteolinidin | —OH | —OH | —H | —OH | —OH | —H | —OH |
| Pelargonidin | —H | —OH | —H | —OH | —OH | —H | —OH |
| Malvidin | —OCH₃ | —OH | —OCH₃ | —OH | —OH | —H | —OH |
| Peonidin | —OCH₃ | —OH | —H | —OH | —OH | —H | —OH |
| Petunidin | —OH | —OH | —OCH₃ | —OH | —OH | —H | —OH |
| Rosinidin | —OCH₃ | —OH | —H | —OH | —OH | —H | —OCH₃ |

Particular preference is given within the context of the invention to a complex with delphinidin.

The invention further provides an aqueous solution of a complex according to the invention.

The invention further provides a process for the preparation of a complex according to the invention and of a corresponding aqueous solution, comprising the steps:
a) preparing an aqueous solution of the methylated β-cyclodextrin,
b) adding the anthocyanidin and mixing to prepare the complex.

In step a), an aqueous solution is preferably prepared which comprises from 10 to 60% by weight, more preferably 20 to 50% by weight, more preferably 30 to 50% by weight, of the cyclodextrin that is used.

It is particularly preferred within the context of the invention if the pH of the aqueous solution is adjusted during or after, but preferably before, the addition of the anthocyanidin, preferably delphinidin, to a pH of 7 or less, preferably 6 to 7. It has been shown that, at this pH, a higher concentration of the complex in aqueous solution can be established.

The concentration of the anthocyanidin, calculated as chloride, is preferably at least 10 mg/ml, more preferably at least 20 mg/ml, more preferably at least mg/ml, more preferably at least 80 mg/ml. Concentrations of about 100 mg/ml can easily be achieved.

The invention likewise provides a solid comprising a complex of an anthocyanidin and a methylated β-cyclodextrin, obtainable by removing the solvent from an aqueous solution. The solvent can be removed from the aqueous solution by methods known to those skilled in the art such as freeze-drying (lyophilization), for example. This solid according to the invention is stable on long-term storage and can be easily mixed again with water to give an aqueous, and therefore an in vivo administrable, solution. Both the aqueous solution and the solid according to the invention have a high storage stability.

Within the context of the preparation according to the invention, mixing of the constituents of the aqueous solution can be carried out by stirring, preferred times for mixing being from 2 to 20 hours. The operation is preferably carried out in the dark in order to avoid light-induced oxidation.

Working examples of the invention are illustrated below.

### 1. Materials Used

RAMEB was acquired from Wacker Chemie and delphinidin chloride from Extrasynthese.

### 2. Determination of the Delphinidin Content

A reversed phase HPLC process was used for determining the content of delphinidin chloride in the delphinidin-containing compositions. The following reagents were used thereby:

Purified water

Methanol for the chromatography

Formic acid, p.a.

1M hydrochloric acid as volumetric solution.

The column used was a Waters X Bridge™ C18, 35 μl, 150 mm×4.6 mm.

The mobile phases were as follows:

Channel A: water 950 ml, methanol 50 ml, formic acid 10 ml

Channel B: water 50 ml, methanol 950 ml, formic acid 10 ml

The following gradient program was used:

| Time [min] | Percent channel B |
|---|---|
| 0 | 0 |
| 5 | 0 |
| 25 | 60 |
| 30 | 100 |

Stop time: 35 minutes

Post time: 8 minutes

Flow rate: 1 ml/min

Injection volume: 20 μl

Column temperature: 30° C.+/−2° C.

UV-Vis detector: 530 μm for the assay, 275 μm for the detection of impurities

Integrator: area

### Solutions and Sample Preparation

Dilution solution 1: mixture of 100 ml of methanol and 2.6 ml of 1M HCl
Dilution solution 2: mixture of 100 ml of 40 percent methanol and 2.6 ml of 1M HCl

Calibration solution: A reference solution of delphinidin was prepared by weighing 10 mg of delphinidin chloride into a 10 ml flask and dissolving it in dilution solution 1. After the dissolution, the solution was diluted approximately 10-fold with dilution solution 2 in order to produce an approximate concentration of 0.1 mg/ml.

The control calibration solution was prepared in the same manner. The calibration solutions were analyzed immediately by means of HPLC because delphinidin chloride is unstable in solution.

### Preparation of the Test Solutions

In order to determine the delphinidin content of solids prepared according to the invention (for preparation see below), approximately 50 mg of this composition were weighed into a 10 ml flask. The composition was then dissolved in dilution solution 2 and diluted further with the same dilution solution 2 until an approximate delphinidin concentration of 0.1 mg/ml was established.

The determination of the delphinidin content in the samples was calculated with the aid of Agilent ChemStation software using calibration with the described external standard.

### EXAMPLE 1

### Complexing of Delphinidin With RAMEB

Solutions of 40% by weight RAMEB in water were prepared.

1 ml of the aqueous cyclodextrin solution was introduced into a glass flask. 250 mg of delphinidin chloride was then added.

The suspension was stirred for 4 hours at 30° C. in the dark. It was then filtered through a membrane filter of 0.8 μm pore size.

### EXAMPLE 2

### Preparation of a Solid According to the Invention

The solution according to example 1 was frozen and then freeze-dried at −48° C. and a pressure of approximately 10.3 Pa (77 mTorr). This gave 0.36 g of a solid with a delphinidin content of 31.1% by weight.

This solid provides delphinidin in high concentration in a storable and readily in vivo administrable form. The delphinidin content of the complex is much higher than in the prior art.

1. A complex of an anthocyanidin and a methylated β-cyclodextrin.

2. The complex as claimed in claim 1, characterized in that the degree of substitution of the β-cyclodextrin with methyl groups is from 10 to 15.

3. The complex as claimed in claim 2, characterized in that the methylated β-cyclodextrin is randomly methylated β-cyclodextrin (RAMEB).

4. The complex as claimed in claim 1, characterized in that the anthocyanidin is selected from the group consisting of aurantinidin, cyanidin, delphinidin, europinidin, luteolinidin, pelargonidin, malvidin, peonidin, petunidin and rosinidin.

5. The complex as claimed in claim 4, characterized in that the anthocyanidin is delphinidin.

6. An aqueous solution of a complex as claimed in claim 1.

7. The aqueous solution as claimed in claim 6, characterized in that the concentration of the anthocyanidin, calculated as chloride, is at least 10 mg/ml.

8. A solid comprising a complex of an anthocyanidin and a methylated β-cyclodextrin, obtainable by removing the solvent from an aqueous solution as claimed in claim 6.

9. A process for the preparation of a complex of an anthocyanidin and a methylated β-cyclodextrin, comprising the steps:
a) preparing an aqueous solution of the methylated β-cyclodextrin,
b) adding the anthocyanidin and mixing to prepare the complex.

10. The process as claimed in claim 9, characterized in that the solution prepared in step a) comprises from 10 to 60% by weight.

11. The process as claimed in claim 9, characterized in that the mixing in step b) takes place over a period of from 2 to 20 hours.

12. The complex as claimed in claim 1, characterized in that the degree of substitution of the β-cyclodextrin with methyl groups is from 11 to 14.

13. The complex as claimed in claim 1, characterized in that the degree of substitution of the β-cyclodextrin with methyl groups is from 12 to 13.

14. The aqueous solution as claimed in claim 6, characterized in that the concentration of the anthocyanidin, calculated as chloride, is at least 20 mg/ml.

15. The aqueous solution as claimed in claim 6, characterized in that the concentration of the anthocyanidin, calculated as chloride, is at least 50 mg/ml.

16. The aqueous solution as claimed in claim 6, characterized in that the concentration of the anthocyanidin, calculated as chloride, is at least 80 mg/ml.

17. The process as claimed in claim 9, characterized in that the solution prepared in step a) comprises from 20 to 50% by weight of the methylated β-cyclodextrin.

18. The process as claimed in claim 9, characterized in that the solution prepared in step a) comprises from 30 to 50% by weight of the methylated β-cyclodextrin.
